(19) United States
(12) Patent Application Publication
(10) Pub. No.: US 2013/0259809 A1
(43) Pub. Date: Oct. 3, 2013

(54) USE OF POLYSACCHARIDES FROM RADIX ISATIDIS IN MANUFACTURE OF MEDICAMENTS AGAINST INFLUENZA VIRUS

(71) Applicants: STATE KEY LABORATORY OF RESPIRATORY DISEASES, Guangzhou (CN); HUTCHISON WHAMPOA GUANGZHOU BAIYUNSHAN CHINESE MEDICINE CO., LTD., Guangzhou (CN)

(72) Inventors: Zifeng YANG, Guangzhou (CN); Chuyuan LI, Guangzhou (CN); Yutao WANG, Guangzhou (CN); Suishan ZHAO, Guangzhou (CN); Nanshan ZHONG, Guangzhou (CN); Qing LIN, Guangzhou (CN); Zheng WANG, Guangzhou (CN); Sheng QIN, Guangzhou (CN); Wenda GUAN, Guangzhou (CN); Ziyao MO, Guangzhou (CN); Deqin WANG, Guangzhou (CN)

(21) Appl. No.: 13/859,138

(22) Filed: Apr. 9, 2013

Related U.S. Application Data
(63) Continuation of application No. PCT/CN2010/001581, filed on Oct. 9, 2010.

(51) Int. Cl. A61K 31/715; C08B 37/00

(52) U.S. Cl. CPC A61K 31/715; C08B 37/0003
USPC 424/43; 514/54; 536/123.1

(57) ABSTRACT

A process of using Isatis root polysaccharide in the manufacture of medicaments for treating and/or preventing diseases induced by influenza virus and complications thereof, wherein the molecular weight of the Isatis root polysaccharide is 3000-7000 Da, and the influenza viruses include influenza virus A, influenza virus B, avian influenza virus, such as human influenza virus H1N1, H3N2, avian influenza virus H6N2, H7N3, H9N2 and INF B. The mechanism of the Isatis root polysaccharide against influenza virus is due to the ability to inhibit the attachment of influenza virus to the host cell. The present invention also discloses a pharmaceutical composition containing the above-mentioned Isatis root polysaccharides and a method for preparing the polysaccharides.

### CROSS REFERENCE TO RELATED APPLICATION

This Application is a continuation application filed under 35 U.S.C. §111(a), claiming the benefit under 35 U.S.C. §120 and §365(c) of the International Application PCT/CN2010/001581, filed Oct. 9, 2010, the disclosure of which is therefore incorporated by reference.

### TECHNICAL FIELD

The present invention belongs to the field of medical technology, specifically, the present invention relates to a pharmaceutical composition for the treatment of diseases caused by influenza virus and method for preparation thereof. The present invention also relates to the use of the pharmaceutical composition in the manufacture of medicaments against influenza viruses, preventive preparations, health foods, and nutrition preparations. The present invention further relates to a method for treating diseases caused by influenza virus and concurrent diseases, disorders, or conditions therewith.

### BACKGROUND ART

Radix Isatidis, having synonyms of isatis root (“Convenient Reader of Materia Medica”), indigowood root (the “Classification of Herbs”), indigo root (“Empirical Identification Method for Shape and Nature of Traditional Chinese Medicine”), is the roots of Isatis tinctoria L. and I. indigotica Fort. of Cruciferae; or the rhizomes and roots of the Strobilanthes cusia of Acanthaceae; or the dried roots of I. indigotica Fort. The property of Radix Isatidis is bitter, cold, and enters heart and stomach meridians. It has efficacy of clearing heat and detoxification, cooling blood, and relieving sore throat. In many traditional Chinese medicine prescriptions, Radix Isatidis has the longest history for clinical treatment of influenza virus.

The existing known component (including indicative component) of Radix Isatidis, such as indoles, indirubin and the like are all not the active components against viruses. The antiviral effect and effective substance of this variety of traditional Chinese medicine has not been confirmed fully so far. The content of polysaccharide from Isatis root in the crude drugs is up to 24.87%, and it is reported that Isatis root polysaccharide has functions of immuno-regulation, anti-tumor and so on. However, during the existing production of various Radix Isatidis preparations, the polysaccharide components are always removed as impurities. The natural components of Isatis root polysaccharide include dextran, rhamnose, arabinose, glucose, galactose, xylose, mannose, lactose, etc. Numerous reports showed that, as a kind of natural low toxic drug, polysaccharides have wide bioactivity of enhanced immuno-regulation, anti-tumor and antivirus, etc., especially with further understanding of its antiviral effect. Natural polysaccharides have become a hot research subject of viral attachment blocker. It is reported that the anionic polysaccharides such as heparin, dextran sulfate, sodium pentosan polysulfate, natural Prunella vulgaris polysaccharide and so on, may have effects on inhibiting animal virus. It is possible to identify the active components from the natural polysaccharides that are capable of inhibiting virus, especially the active components that are capable of blocking virus attachment and it has lasting values of research and development. Sialic acid receptor on the surface of the host cells of influenza virus is an oligosaccharide terminated by sialic acid, and the constituents of which are galactose, N-acetyl glucosamine, N-acetyl galactosamine. Since the monosaccharide types of Isatis root polysaccharide seem similar to components of sialic acid receptor, Isatis root polysaccharide components may simulate the conformation of sialic acid receptor and interact with viral RBS, thus inhibiting virus from recognizing and binding sialic acid receptor. In currently disclosed methods related to development and utilization of Radix Isatidis, there are no reports on using the prepared polysaccharides for inhibiting various influenza virus subtypes, especially inhibiting attachment of influenza virus to block infection.

Anti-viral drugs that currently can be used for treating and preventing influenza virus are mainly neuraminidase inhibitors (NAI), such as oseltamivir and zanamivir; and amantadines, which are M2 ion channel inhibitors, including amantadine and rimantadine. However, the two types of drugs as described above have various side effects and limitation, e.g. tendency to develop resistance, etc., which also become the important problems of clinical treatment and new drug development. Whereas the antiviral herbs have advantages of stable efficacy, low toxic and side effect, and being less tendency to develop resistance, not promoting virus mutation, as well as have abundant medicinal herbs resources and low cost, therefore, the antiviral herbs play an irreplaceable role in treating viral diseases based on the unique advantage of efficacy, particularly when human being face long term and tough challenges of viral diseases, the antiviral herbs will have huge market and wide prospect of development. Radix Isatidis is one of the herbs that exhibit anti-viral effect.

In conclusion, there is now a need for the investigation of whether the Isatis root polysaccharide, especially polysaccharides that are discarded during the production of Radix Isatidis preparations inhibit viruses and its inhibition mechanism and uses thereof, and thus there is also a need for investigation and use of the pharmaceutical composition comprising the Isatis root polysaccharide as an active ingredient.

### DISCLOSURE OF THE INVENTION

In order to solve the technical problems mentioned above, the present invention uses Radix Isatidis for isolation of the active polysaccharides, specifies the effective antiviral components and antiviral mechanism thereof, which provide valuable references for the production process of Radix Isatidis and also broaden ways of development and utilization of the natural products of Radix Isatidis.

To facilitate the understanding of the present invention, some terms are defined below. Terms defined herein have meanings as commonly understood by a person of ordinary skill in the art relevant to the present invention. Unless otherwise specified, the term “Isatis root polysaccharide” as used herein refers to the total polysaccharide from Radix Isatidis, formed by condensation and dehydration of many monosaccharide molecules, is a kind of carbohydrates with complex and huge molecular structures.

Unless otherwise specified, the term “active polysaccharide” as used herein refers to the polysaccharide compounds having certain special physiological activity, which have the bidirectional regulation function of circadian rhythm of human body. It has very important and special physiological activity, participates in the immunoregulation of organism and various activities of living cells, is a high molecular polymer formed by an aldehyde group and a ketone group which join together via glycosidic bonds.

Unless otherwise specified, the term “pharmaceutically acceptable carrier and excipient” as used herein refers to those substances that are used as a filler or carrier in the pills, tablets, capsules, injections and the like as is well known in the art. These substances are usually recognized to use for this purpose and as the non-active ingredient of the preparations by healthcare professionals. A list regarding pharmaceutically acceptable carries and excipients can be found in reference books, such as Handbook of Pharmaceutical excipients, version 2, edited by A. Wade and P. J. Weller; published in American Pharmaceutical Association, Washington and The Pharmaceutical Press, London, 1994.

Unless otherwise specified, the term “therapeutically effective amount” as used herein refers to an amount of the drugs that is required for producing effective action, which can be altered and is finally determined by the medical personnel. The considered factors include route of administration, property of preparations, general condition such as weight and age of the subject, nature and severity of diseases to be treated and the like.

Unless otherwise specified, the term “active ingredient” as used herein refers to components having active effect or certain treating/preventing effect, or components with a certain weight and having active effect or certain treating/preventing effect.

Unless otherwise specified, the term “dosage form” as used herein refers to a well known dosage form in the art, in which pharmaceutical preparation or composition is prepared as a separate administration unit form with each unit usually containing single dose or several doses of administration according to the actual situation.

An object of the present invention is to provide a new medical use of Isatis root polysaccharide in treating diseases caused by influenza viruses and complications thereof. Another object of the present invention is to provide the use of Isatis root polysaccharide in the manufacture of medicaments for preventing and/or treating diseases caused by influenza viruses and complications thereof. Yet another object of the present invention is to provide a composition comprising Isatis root polysaccharide used for acting against influenza viruses and uses thereof. A further object of the present invention is to provide a method for treating diseases caused by influenza viruses and concurrent diseases, disorders, or conditions therewith.

Regarding the above-mentioned objects, the present invention provides the following technical solutions:

In one aspect, the present invention provides a process of using Isatis root polysaccharide in preventing and/or treating diseases caused by influenza viruses and complications thereof, wherein the molecular weight of the Isatis root polysaccharide is 3000-7000 Dalton.

The Isatis root polysaccharide is extracted from Radix Isatidis, preferably prepared by a method comprising the following steps:

A. decocting the Radix Isatidis raw materials by adding suitable times of distilled water to produce a decocted fluid, and concentrating the decocted fluid so that the baume degree of which is 18-20° Bé at 50° C.;

B. cooling the concentrated solution obtained in Step A to below 45° C., adding alcohol to produce an alcohol solution so that alcohol content is up to 60% or above 60% in the concentrated solution, allowing the solution to stand for more than 12 hours to precipitate;

C. passing the alcohol solution through a macroporous resin column, eluting with pure water, and collecting the eluent;

D. deproteinizing the eluent with methods known in the art, then placing the deproteinized solution in a dialysis bag with a proper molecular weight to dialyse; and

E. evaporating the solution inside and/or outside the dialysis bag with a rotary evaporator to concentrated solution, and then freeze-drying the resulted components with a vacuum freeze drier to powder to obtain the powder components of the Isatis root crude polysaccharide.

Preferably, the influenza viruses include but are not limited to influenza A, B viruses and avian influenza virus; more preferably, the influenza viruses include but are not limited to human influenza virus subtypes H1N1 (including novel influenza A virus H1N1), H3N2, avian influenza virus subtypes H6N2, H7N3, H9N2, and INF B.

In another aspect, the present invention provides a process of using the above-mentioned Isatis root polysaccharide in the manufacture of medicaments for preventing and treating diseases caused by influenza viruses and complications thereof, wherein the molecular weight of the Isatis root polysaccharide is 3000-7000 Dalton.

Preferably, the influenza viruses include but are not limited to influenza A, B viruses and avian influenza virus; more preferably, the influenza viruses include but are not limited to human influenza virus subtypes H1N1 (including novel influenza A virus H1N1), H3N2, avian influenza virus subtypes H6N2, H7N3, H9N2, and INF B.

Preferably, dosage forms of the medicament include but are not limited to oral preparations, parenteral preparations, topical and inhaled preparations and transdermal preparations;

More preferably, dosage forms of the medicament include but are not limited to aerosols, capsules, eardrops, eye drops, eye ointments, gels, granules, injections, liniments, lotions, nasal drops, ointments, oral preparations, patches, pellicles, powders, solutions, suppositories, syrups, tablets, and tinctures etc.

The present invention further provides the use of Isatis root polysaccharide in the manufacture of health foods or nutrition preparations for preventing diseases caused by influenza viruses and complications thereof, wherein the molecular weight of the Isatis root polysaccharide ranges from 3000 to 7000 Dalton.

Preferably, the influenza viruses include but are not limited to influenza A, B viruses; more preferably, the influenza viruses include but are not limited to human influenza virus H1N1 subtypes (including novel influenza A virus H1N1), H3N2, avian influenza virus subtypes H6N2, H7N3, H9N2, and INF B.

In yet another aspect, the present invention provides a pharmaceutical composition for preventing and/or treating diseases caused by influenza viruses and complications thereof, wherein the pharmaceutical composition comprises Isatis root polysaccharide, and wherein the molecular weight of the Isatis root polysaccharide ranges from 3000 to 7000 Dalton.

Preferably, the influenza viruses include but are not limited to influenza A, B viruses and avian influenza virus; more preferably, the influenza viruses include but are not limited to human influenza virus subtypes H1N1 (including novel influenza A virus H1N1), H3N2, avian influenza virus subtypes H6N2, H7N3, H9N2, and INF B.

Preferably, the pharmaceutical composition comprises one or more pharmaceutically acceptable carriers or excipients in addition to the Isatis root polysaccharide as an active ingredient.

Preferably, the pharmaceutical composition includes but is not limited to oral preparations, parenteral preparations, topical and inhaled preparations, and transdermal preparations.

More preferably, the pharmaceutical composition includes but is not limited to aerosols, capsules, eardrops, eye drops, eye ointments, gels, granules, injections, liniments, lotions, nasal drops, ointments, oral preparations, patches, pellicles, powders, solutions, suppositories, syrups, tablets, and tinctures etc.

Preferably, carriers or excipients in the pharmaceutical composition include but are not limited to diluents, adhesives, disintegrants, lubricants, substrates, aromatics, sweeteners, colorants, preservatives, antioxidants, coating agents, film-forming materials, solvents, solubilizers, wetting agents, adsorbents, filter aids, emulsifiers, surfactants, suspending agents, thickeners, plasticizer, chelating agents, transdermal enhancers, aerosol propellants, foaming agents, acidifying or alkalizing agents, buffers and the like.

In another aspect, the present invention provides a method for preparing the Isatis root polysaccharide as follows:

A. Decocting the Radix Isatidis raw materials by adding suitable times of distilled water to produce a decocted fluid, and concentrating the decocted fluid so that the baume degree of which is 18-20° Bé at 50° C.

B. Cooling the concentrated solution obtained in Step A to below 45° C., adding alcohol so that alcohol content is up to 60% or above 60% in the concentrated solution, allowing the solution to stand for more than 12 hours to precipitate.

C. Passing the alcohol solution through a macroporous resin column, eluting with pure water, and collecting the eluent.

D. Deproteinizing the eluent with methods known in the art, then placing the deproteinized solution in a dialysis bag with a proper molecular weight to dialyse.

E. Evaporating the solution inside and/or outside the dialysis bag with a rotary evaporator to concentrated solution, and then freeze-drying the resulted components with a vacuum freeze drier to powder to obtain the powder components of the Isatis root crude polysaccharide with proper molecular weight.

Preferably, the method for preparing the Isatis root polysaccharide provided in the present invention comprises the following steps:

A. Decocting the Radix Isatidis raw materials by adding 8 times amount of distilled water for 2 hours at the first time to obtain the decocted fluid A and draff of Radix Isatidis; filtering the decocted fluid A and pouring into another container, adding 8 times amount of water into the draff and continue to decoct for 4 hours to obtain the decocted fluid B of Radix Isatidis.

B. Filtering the decocted fluid B and combining the filtrates with that of the decocted fluid A, concentrating to concentrated solution of Radix Isatidis, determining the baume degree of the concentrated solution with a baume hydrometer at 50° C., which is 18-20° Bé, and then cooling.

C. After cooling the concentrated solution of Radix Isatidis to a temperature below 45° C., adding alcohol so that alcohol content is up to 60% or above 60% in the concentrated solution, allowing the solution to stand for more than 12 hours to precipitate.

D. Passing the alcohol solution through a macroporous resin column, eluting with pure water, and collecting the eluent.

E. Deproteinizing the Isatis root crude polysaccharide solution in pure water using sevage method: based on the property that proteins will denature in the organic solvent such as chloroform, shaking the mixture of sevag reagent and Isatis root crude polysaccharide solution in pure water violently with the ratio of sevag reagent (chloroform: n-butyl alcohol=5:1): Isatis root crude polysaccharide being 5:1 for 20 to 30 min to allow the proteins and chloroform-n-butyl alcohol generating gel so as to isolate the proteins from the Isatis root crude polysaccharide solution in pure water. Then centrifuging (4000 rpm/min×10min) to remove the denatured proteins at the interface of aqueous layer and solvent layer, repeating for 5 times until there is no obvious denatured proteins precipitating out.

F. Placing the aqueous layer in a dialysis bag with different molecular weight and then placing the dialysis bag holding the Isatis root crude polysaccharide solution in flask, adding tri-distilled water, and stirring for 24 hours with a magnetic stirrer.

G. Evaporating the solution inside and/or outside the dialysis bag with a rotary evaporator to concentrated solution, and then freeze-drying the resulted components with a vacuum freeze drier to powder to obtain the powder components of the Isatis root crude polysaccharide with proper molecular weight.

The present invention further provides a method for preparing the pharmaceutical composition, wherein the method includes using the Isatis root polysaccharide with a molecular weight of 3000-7000 Dalton as an active ingredient, adding auxiliary materials according to pharmaceutical formulations, and preparing preparations using the conventional technique in pharmaceutics.

In yet another aspect, the present invention further provides a method for treating diseases caused by influenza viruses and complications thereof, characterized in administering therapeutically effective amounts of pharmaceutical composition mentioned above or administering therapeutically effective amounts of Isatis root polysaccharide to the subject, wherein the molecular weight of the Isatis root polysaccharide is 3000-7000 Dalton.

To illustrate and set out the technical solution of the present invention more clearly, the present invention is further described below:

According to the systematic study of the anti-influenza effect of Isatis root polysaccharide by the applicant, it is found that the polysaccharide haves good anti-influenza effect, mainly against influenza A, B viruses, wherein subtypes of the influenza A virus include human influenza virus subtypes H1N1 (including novel influenza A virus H1N1), H3N2, avian influenza virus subtypes H6N2, H7N3, H9N2, whereas it has no anti-influenza effect on other respiratory viruses, such as respiratory syncytial virus, adenovirus, parainfluenza virus and the like. The anti-influenza virus mechanism is blocking the process of viral infection by acting on the early attachment stage of influenza virus replicative cycle. Therefore, the present invention provides the pharmaceutical use of the Isatis root polysaccharide having anti-influenza effect.

The Isatis root polysaccharide of the present invention can be used as raw materials of antiviral agents and preventive agents in pharmaceutical industry.

The composition or anti-viral agents and preventive agents, preferably in forms of preparation, of the present invention can be prepared using the well-known methods in the art. These types of methods include the step of mixing the active ingredients with the carriers making up one or more auxiliary components. These types of auxiliary components include common components in the art, such as fillers, binders, diluents, disintegrants, lubricants, coloring agents, flavoring agents and wetting agents.

According to the drugs and pharmaceutical components, the preparations of the present invention can be prepared by using known means, device, method and process and the like in the pharmaceutical preparation technology. For example, for solid preparation such as tablet, the active ingredient, i.e. Isatis root polysaccharide can be mixed evenly with the pharmaceutical carrier, for example, the common components of tablet such as corn flour, lactose, sucrose, sorbitol, talc, magnesium stearate, dicalcium phosphate or pharmaceutically acceptable colloid and then tableted together, to form an solid component comprising evenly distributed Isatis root polysaccharide. Even distribution herein can be understood as uniform distribution of the active ingredient Isatis root polysaccharide in the whole components, thus it can be easily divided into unit dosage form having the same activity. Pharmaceuticals or pharmaceutical components of the present invention may also be coated or mixed with another component to provide a slow release formulation, in which suitable coated components include but are not limited to polyacid and mixture of polyacid with materials e.g. shellac, cetyl alcohol and/or cellulose acetate etc.

The pharmaceutical composition of the present invention may also be prepared as other clinically acceptable preparations, e.g. granules, capsules, dropping pills, subcutaneous preparations and the like. All these dosage forms can be prepared in accordance with methods well known by those skilled in the art. For example, when preparing granules, the pharmaceutical excipients required include but are not limited to dextrin, starch, lactose, glucose, mannitol, sodium carboxymethyl cellulose and surfactant that can be used for increasing the stability of the drugs; when preparing capsules, the common encapsulating materials in the art can be used; when preparing dropping pills, water soluble auxiliary materials are required, for example polyethylene glycols, such as polyethylene glycol 6000, polyethylene glycol 4000, polyethylene glycol 300 and soaps; when preparing subcutaneous preparations, such as injections, proper pH regulators and preservatives can be selected as the auxiliary materials as required.

Comparing with the state of the art, the present invention has the following advantages:

Firstly, during the existing production of various preparations of Radix Isatidis, polysaccharide components have been removed as impurities all along and have not been utilized. The present invention demonstrates through investigation that Isatis root polysaccharide extracted from Radix Isatidis, particularly Isatis root polysaccharides with the molecular weight of 3000-7000 Dalton have anti-influenza effect and can be used to prepare medical preparations or health products having preventive effect, broadening the range of development and application of the Chinese traditional medicine Radix Isatidis;

Secondly, in the currently disclosed development and utilization of Radix Isatidis, it has not been reported that active polysaccharides prepared from Radix Isatidis are used for inhibiting a variety of influenza subtypes, especially inhibiting the attachment of influenza viruses. After extracting the active polysaccharides from Radix Isatidis, the present invention defines the effective anti-viral components and mechanism of anti-viral activities of the active polysaccharides, which has important reference value for clinical application of Radix Isatidis.

Thirdly, in currently existing methods and preparations for the treatment and prevent of influenza, there is a technical defect of extracting and isolating the active substances, while in the present invention, active polysaccharides are extracted from the Chinese traditional medicine, Radix Isatidis, as the active ingredients against influenza virus, having the advantages of being more safety and stability in clinical application and health care.

### DESCRIPTION OF THE DRAWINGS

Examples of the present invention are described in detail in conjunction with the drawings as following, in which:

FIG. 1 is the isolation technology route and activity screening of Isatis root polysaccharide; and

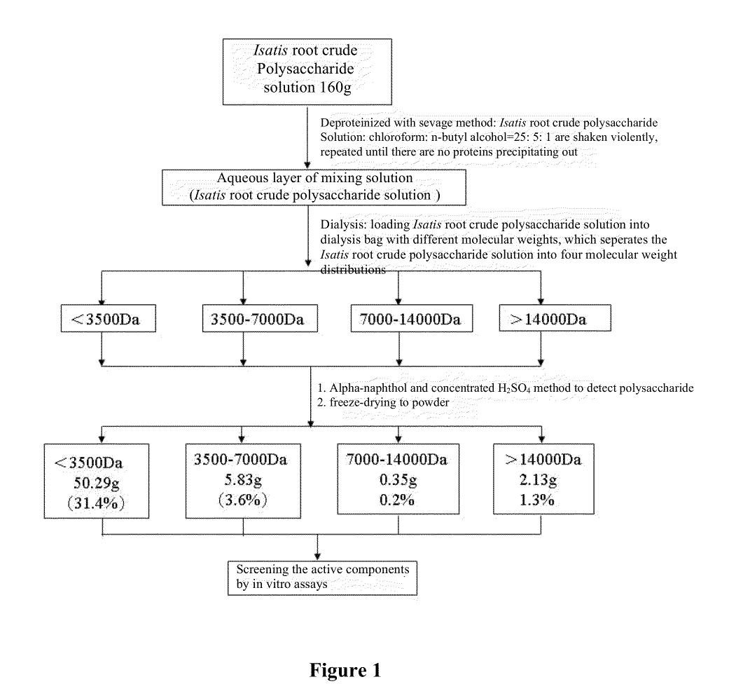

FIG. 2 is the inhibitory effect of Isatis root polysaccharide component G2 (3500-7000 Da) on blood coagulation of different influenza viruses.

### BEST MODE FOR CARRYING OUT THE INVENTION

The present invention is now further set forth in conjunction with specific examples. But these examples are only used to illustrate the present invention and do not limit the scope of the present invention. In the following examples, the experimental methods that do not indicate the specific experimental conditions are usually according to conventional conditions or according to the conditions proposed by the manufacturer.

The common used instruments and their sources in this study:

| Instruments | Types | Manufacturer or Company |
|---|---|---|
| Vacuum Freeze Dryer | ALPHA 1-2LD PLUS | CHRIST, Germany |
| Vortex micro-mixer | MS1 | IKA, Germany |
| Double beam UV-visible light | TU-1901 | Beijing Purkinje General Instrument |
| spectrophotometer | | Co., Ltd |
| Dialysis bag | 27 mm [MW: 3500] | US |
| | 27 mm [MW: 7000] | Agent: Shanghai Bioscience |
| | 36 m [MW: 8000-14000] | Technology Co. Ltd. |
| High performance liquid | Agilent 1200 | Agilent Technologies, US |
| chromatograph | | |
| Variable wavelength detector | VWD-G1314B | Agilent Technologies, US |
| Evaporative Light- | ELSD2000 | ALLTECH, Germany |
| scattering Detector | | |
| ZORBAX Eclipsc XDB-C18 | 4.6 × 150 nm, 5 μm | Agilent Technologies, US |
| SRT SEC-150 | 300 mm × 7.8 mm 5 μm 150 A | Sepax Technologies, Inc., US |
| Packing material of | Sephadex-G50 | GE Guangzhou Sepol instrument Co. |
| chromatographic column | | Ltd. |

The common used reagents and their sources in this study:

| Reagents | Manufacturer or Company |
|---|---|
| 5%NaHCO₃ Silica gel plate, | Dr. Ehrenstorfer gmbh, |
| D (+) - Galactose, D (+) - Xylose, L (+) - | Germany |
| Arabinose, D (+) -Glucose, L (+) - | Purchased from Guangzhou |
| Rhamnose Hydrate | Sepol Laboratory Equipment |
| | Co. Ltd. |
| D-Glucuronic Acid | Tianjin yifang Technology |
| | Co. Ltd. |
| | Purchased from Guangzhou |
| | Sepol Laboratory Equipment |
| | Co. Ltd. |
| Dextran T-3, Dextran T-4, Dextran T-6, | US |
| Dextran T-7, Dextran T-9 | Purchased from Nanjing Duly |
| | Biotech Co., Ltd |
| Dextran T-5, DextranT-10 | Pharmacia, Sweden |
| | Nanjing Duly Biotech |
| | Co., Ltd |

The common used cells, viruses, herbs and their sources in this study:

| Cells, viruses and herbs | Manufacturer or Company |
|---|---|
| Canine kidney cells (MDCK) | Cell Bank, Committee on |
| GN023 | Type Culture Collection |
| | of Chinese Academy of |
| | Sciences |
| Influenza A virus subtype H1N1, strain | American Type Culture |
| PR8 (A/PR/8/34, H1N1) VR-1469 | Collection (ATCC) |
| Influenza A virus subtype H1N1, strain | |
| FM1 (A/FM1/47, H1N1) VR-97 | |
| Influenza A virus subtype H3N2, strain | |
| Aichi (A/Aichi/2/68, H3N2) VR-0547 | |
| Novel Influenza A virus subtype H1N1 | Clinical isolates |
| (A/Guangzhou/GIRD07/09, H1N1, | in this lab |
| Genebank No. HM014332.1) | |
| Seasonal Influenza A virus subtype H1N1 | |
| strain (A/Guangzhou/GIRD02/2009, H1N1) | |
| Influenza B virus (B/Guangzhou/ | |
| GIRD08/09) | |
| Influenza A virus subtype H6N2 | Kindly privided by |
| (A/Duck/Guangdong/2009, H6N2) | Professor Chen Jianxin, |
| H7N3 (A/Duck/Guangdong/1994, H7N3) | Veterinary schools, South |
| H9N2 (A/Chicken/Guangdong/1996, H9N2) | China Agriculture |
| | University |
| Radix Isatidis herbs | Origin of GAP Herbs: |
| | Fuyang, Anhui Province |

### EXAMPLE 1

### Preparation of Isatis Root Polysaccharide Powder

Isatis root polysaccharide powder, which will be determined for antiviral activity in vitro, is prepared according to the following method:

1. Preparation of Radix Isatidis Aqueous Extract

(1) 10 kg of Radix Isatidis herbs are decocted with 8 times amount of water for 2 times, 2 hours for the first time and 4 hours for the second time to obtain the decocted fluid of Radix Isatidis.

(2) The decocted fluid of Radix Isatidis is filtered, combine the filtrate and concentrate to 18-20° Bé (determined at 50° C.) to get the concentrated solution of Radix Isatidis.

(3) The concentrated solution of Radix Isatidis is cooled to a temperature below 45° C., alcohol is added so that alcohol content is up to 60%, and allows standing for more than 12 hours to precipitate.

(4) The alcohol solution is passed through a macroporous resin column, eluted with different polar solvents such as pure water, 20% alcohol, 40% alcohol, 60% alcohol, 80% alcohol, 100% alcohol successively to get the eluent of different polar solvents.

(5) The eluent of different polar solvents is determined for antiviral activity in vitro by cytopathic effect inhibition method (CPE method) to come to the conclusion that the active ingredient is contained in water eluent (S-03).

(6) The water eluent which is prepared according to the above method is the Isatis root crude polysaccharide solution.

2. Isolation of Isatis Root Polysaccharide

(1) The Isatis root crude polysaccharide solution in which the content of polysaccharide is 70% is deproteinized using sevage method. Based on the property that proteins will denature in the organic solvent such as chloroform, the Isatis root crude polysaccharide in pure water is shaken violently with the ratio of sevag reagent (chloroform: n-butyl alcohol=5:1): Isatis root crude polysaccharide being 5:1 for 20 to 30 min to allow the proteins and chloroform-n-butyl alcohol generating gel so as to isolate the proteins from the Isatis root crude polysaccharide solution in pure water. Then centrifuging (4000 rpm/min×10 min) to remove the denatured proteins at the interface of aqueous layer and solvent layer, repeating for 5 times until there is no obvious denatured proteins precipitating out.

(2) The aqueous layer is placed in dialysis bags with different molecular weights which are: <3500 Da, 3500-7000 Da, 7000-14000 Da and >14000 Da respectively. The dialysis bags holding Isatis root crude polysaccharide is placed in flask, tri-distilled water is added and stirred for 24 hours with a magnetic stirrer.

(3) To each 2 mL of the solution inside and outside the dialysis bag, 1.0 mL of alpha-naphthol solution with the mass concentration of 0.05 g/mL is added, shaked up, and then 5.0 mL of concentrated H₂SO₄ is added vertically and quickly, shaked well, after allowing to stand for 5 min, color change is observed. After reaction, purple rings between layers of alpha-naphthol and concentrated H₂SO₄ can be observed, indicating this solution contains carbohydrates.

(4) The crude polysaccharide solutions inside and outside the dialysis bag are concentrated using rotary evaporators respectively to concentrated solution, and then four components with different molecular weight are freeze-dried with vacuum freeze driers to powder to obtain the powder of the Isatis root crude polysaccharide with different molecular weight, including powder G1 (<3500 Da), G2 (3500-7000 Da), G3 (7000-14000 Da) and G4 (>14000 Da).

### EXAMPLE 2

### Preliminary Screening of Antiviral Activity In Vitro of Different Components of Isatis Root Polysaccharide

Preliminary screenings of four components with different molecular weight are conducted for antiviral activity in vitro.

### 1. Experimental Materials

As described previously (cells, viruses, herbs and their sources)

### 2. Experimental Method

The inhibitory effect of different components of Radix Isatidis on influenza viruses is further confirmed using cytopathic effect inhibition method under three experimental strategies of treatment, protection and viral attachment.

### (1) Treatment Mode:

In 24-well cell culture plate, MDCK cells are cultured with MEM medium containing 10% inactivated serum (FBS) till the cells grow to 80% confluency;

The positive control drug (ribavirin) and drugs with different determined concentrations, i.e. Isatis root polysaccharide with different molecular weight of Example 1 are set;

Attachment of virus to cells is performed for 2 hours under the condition of 37° C., 5% CO₂;

The positive drug (ribavirin), test drug with different concentration (initial concentration G1-4: 10 mg/mL, 2 times dilution) (except for the negative control) are added into MDCK cells in 24-well cell culture plate, and then cultured under the condition of 37° C., 5% CO₂.

### (2) Protection Mode:

Culturing MDCK cells in 24-well cell culture plate (similar to the cells and their culture condition used in the treatment mode);

Adding drugs (similar to the positive drugs, test drugs and their concentration used in the treatment mode) into the cultured MDCK cells, incubating for 2 hours;

Performing the attachment of virus to cells for 2 hours (similar to the virus and experimental condition used in the treatment mode);

Replacing the MEM medium with serum free MEM containing TPCK pancreatin, and culturing under the condition of 37° C., 5% CO₂.

### (3) Direct Action Mode:

Viruses and different concentration of drugs are incubated at 37° C. for 1 hour, and then inoculated in cells, incubated at 4° C. for 1 hour, the supernatant is removed and replaced with serum free MEM containing TPCK, cultured at 37° C. for 8 hours.

After culturing 24 and 48 hours under different modes, cytopathic effect (CPE) is observed under inverted microscope. Positive drug control, normal cells control and virus control are set in experiment. If “++” occurs in the virus control, the experiment is ended.

The cytopathic effect degree is recorded by the following 6-grade standards: − represents the cells growing normally, with no cytopathic effect appearing; ± represents the cytopathic effect being less than 10% of the whole monolayer cells; + represents the cytopathic effect accounting for about 25% of the whole monolayer cells; ++ represents cytopathic effect accounting for about 50% of the whole monolayer cells; +++ represents the cytopathic effect accounting for about 75% of the whole monolayer cells; ++++ represents the cytopathic effect accounting for above 75% of the whole monolayer cells. The half inhibitory concentration (IC₅₀) is calculated by Reed-Muench method and expressed as selection index, SI (_{SI=TC}₅₀/IC₅₀, TC₅₀ is half toxic concentration of drug), SI>2 represents low toxicity and high effectiveness; SI: 1˜2 represents high toxicity and low effectiveness; SI<1 represents no effectiveness.

### 2. Results of Preliminary Screening of In Vitro Anti-Influenza Viral Activity

The inhibitory effect of different components of Isatis root polysaccharide (G1, G2, G3 and G4) on influenza viruses is further confirmed using cytopathic effect inhibition method under three experimental strategies of treatment, protection and viral attachment. Results showed that only G2 has obvious in vitro antiviral effect.

| Name of | | A | | B | | C | |
|---|---|---|---|---|---|---|---|
| Samples | TC50 | IC₅₀ | SI | IC₅₀ | SI | IC₅₀ | SI |
| G1 | 27.5 | >10 | <1 | 10 | <1 | >10 | <1 |
| G2 | 14.5 | >10 | <1 | 2.5 | 5.8 | 0.625 | 23.2 |
| G3 | 6.9 | >10 | <1 | >10 | <1 | >10 | <1 |
| G4 | 10.0 | >10 | <1 | >10 | <1 | >10 | <1 |

Selection index (SI), SI > 2 represents low toxicity and high effectiveness;
SI = 1~2 represents high toxicity and low effectiveness;
SI < 1 represents no effectiveness.
A: protection mode;
B: treatment mode;
C: direct mode;

### EXAMPLE 3

### Toxicity Test of Isatis Root Polysaccharide Component G2 (MTT Method)

### 1. Experimental Method

MDCK cells are prepared conventionally, inoculated into a 96-well plate. After 24 hours, i.e. the cells grow to monolayer, the culture solution is removed, and toxicity test well, blank control well and normal cell controls well are set. In addition to cell culture solution, materials added into each wells are as follows:

(1) Blank well: no cell grows, adding 100 μL/well MEM;

(2) Normal cell control well: normal cells grow, adding 100 μL/well MEM, drug dissolution medium with same concentration (MEM);

(3) Toxicity test well: normal cells grow, adding 100 μL/well Isatis root polysaccharide component G2 with different dilution (2 times dilution), continue to culture for 36-48 hours at 37° C., 5% CO₂, after which each well is added with 20 μL MTT solution (5 mg/mL), placed in incubator at 37° C., 5% CO₂, and continue to incubate for 4 hours. The cultured supernatant is removed, and then each well is added with 100 μL of dimethyl sulfoxide (DMSO), vibrated under low velocity for 10 min to make the crystal substance dissolving sufficiently. Each of the wells is measured for absorption value at 570 nm of wavelength on ELISA Reader. Inhibitory rate are calculated according to the following formula:

Inhibitory rate=[(normal-blank)−(administration-blank)]/(normal-blank)×100%

And the 50% toxic concentration is calculated by Reed-Muench method as half toxic concentration of drug (TC₅₀).

### 2. Results of Toxicity Test on Drugs

Toxicity test of the drug sample on viral host cells is a prerequisite for the antiviral efficacy evaluation, the half toxic concentration of drug (TC₅₀) of the test sample G2 is determined using MTT method as 14.5 mg/mL.

### EXAMPLE 4

### Confirmation of Antiviral Efficacy Against Influenza Virus of Isatis Root Polysaccharide Component G2

### 1. Experimental Method

The inhibitory effect of Isatis root polysaccharide component G2 on influenza viruses is further confirmed using Plaque Reduction Assay under three experimental strategies of treatment, protection and viral attachment.

1.5 mL of agarose nutrient containing 0.2% BSA, 0.8% Agar and 0.3% DEAE-Dextran is added on the monolayer cells which are subjected to different modes of treatment (as above described), cultured for 3 days under the condition of 34° C., 5% CO₂, fixed and strained with formalin buffered crystal violet solution (0.1%), and calculate the plaque formation unit (PFU/mL). The half inhibitory concentration (IC₅₀) is calculated by Reed-Muench method and expressed as selection index, SI (SI=TC₅₀/IC₅₀).

### 2. Results of Antiviral Efficacy Against Influenza Virus

The inhibitory effect of Isatis root polysaccharide component G2 on influenza viruses is further confirmed using Plaque Reduction Assay under three experimental strategies of treatment, protection and viral attachment. The results are shown in Table 1:

TABLE 1
Efficacy of Isatis root polysaccharide
component G2 on influenza viruses

| Subtypes of virus | Strains | IC₅₀ | Selection index (SI) |
|---|---|---|---|
| H1N1 | (PR8) | 0.39 ^{c} | 37 |
| H1N1 | (FM1) | 2.52 ^{c} | 5.75 |
| H1N1 | (S-OIV) | 4.3 ^{c} | 3.4 |
| H1N1 | (Isolate) | 1.25 ^{c} | 11.6 |
| H3N2 | (Aichi) | 2.85 ^{c} | 5 |
| H6N2 | (GD) | 5 ^{c} | 2.9 |
| H7N3 | (GD) | 3.96 ^{c} | 3.6 |
| H9N2 | (GD) | 3.91 ^{c} | 3.7 |
| INF B | (Isolate) | 3.63 ^{c} | 4 |

A: protection mode;
B: treatment mode;
^{c} direct mode
SI > 2 represents low toxicity and high effectiveness; SI = 1~2 represents high toxicity and low effectiveness; SI < 1 represents no effectiveness

### EXAMPLE 5

### Hemagglutination Inhibition Experiment for Influenza Virus of Isatis Root Polysaccharide Example 5 Hemagglutination Inhibition Experiment for Influenza Virus of Isatis Root Polysaccharide Component G2

1. Experimental method

A certain amount of anticoagulant chicken blood is washed for 3 times with saline, formulated with sterile saline to 0.5% suspension of chicken erythrocytes. 100 μL of diluted (2 times) G2 sample is added to 96-well plate and 50 μL influenza virus suspension containing 4 hemagglutination units (HAU) is added to each well. After allowing standing at 4° C. for 45 min, 0.5% chicken erythrocytes suspension is added, shaked up and placed in 4° C. refrigerator, and the result is observed after 30-60 min.

### 2. Results of Hemagglutination Inhibition Experiment for Influenza Virus

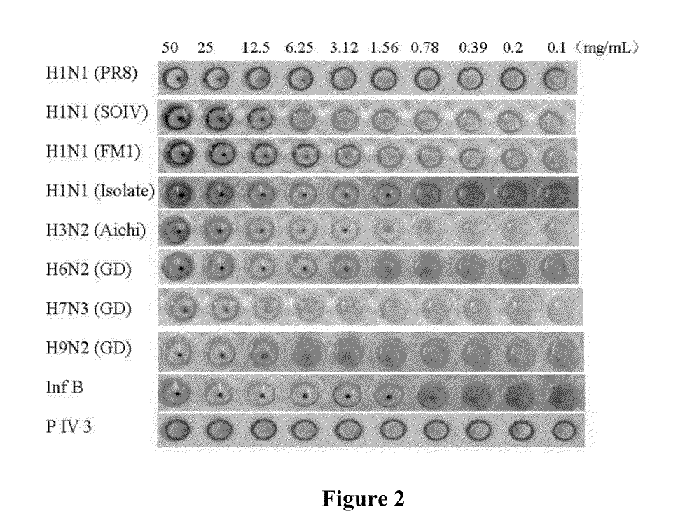

The experimental results are shown in FIG. 2.

G2 has different degrees of inhibitory effect on hemagglutinin of different subtypes of influenza virus strain in hemagglutination inhibition experiment, while it has no inhibitory effect on parainfluenza virus which also has hemagglutinin, indicating that it has specific effect on influenza virus.

### EXAMPLE 6

### Inhibitory Effect of Isatis Root Polysaccharide Component G2 on Other Respiratory Viruses (Non-Influenza Viruses)

### 1. Materials and Experimental Method

(1) Cells:

Canine kidney cells(MDCK), human laryngeal carcinoma cells (HEp-2), monkey kidney cells (LLC-MK₂) are introduced from American Type Culture Collection (ATCC) and Cell Bank, Committee on Type Culture Collection of Chinese Academy of Sciences respectively.

(2) Virus Strain

Adenovirus (ADV), respiratory syncytial virus (RSV), parainfluenza virus 3 (PIV-3), purchased from ATCC.

(3) Experimental Method

The corresponding host cells are infected with each virus in an amount of 100TCID₅₀, 100 μL of multiple proportion (2 times) dilution of Isatis root polysaccharide component G2 is added, placed at 5% CO₂, 37° C. for 48 hours, after which the results are observed. The cytopathic effect degree is the same as Example 1, and the half inhibitory concentration (IC₅₀) is calculated by Reed-Muench method.

### 2. Results of Inhibitory Effect on Other Respiratory Viruses (Non-Influenza Viruses)

The results are shown in Table 2.

TABLE 2
Inhibitory effect of Isatis root polysaccharide
component G2 on other respiratory viruses

| | | | Isatis root polysaccharide | |
|---|---|---|---|---|
| Types of viruses | | Host cells | component G2 | |
| DNA | Adenovirus | HEp-2 | IC₅₀ | >10 |
| virus | (ADV) | | SI | <1 |
| RNA | Respiratory | HEp-2 | IC₅₀ | >10 |
| virus | syncytial virus | | SI | <1 |
| | (RSV) | | | |
| | Parainfluenza virus 3 | | IC₅₀ | >10 |
| | (PIV-3) | LLC-MK2 | SI | <1 |

The experimental results indicate that antiviral effect of Isatis root polysaccharide component G2 shows up as direct action mode, has obvious inhibitory effect in vitro on various subtypes of influenza virus, and has different degrees of inhibitory effect on hemagglutinin of various subtypes of influenza virus, thus inhibiting the attachment of viruses to host cells, indicating good antiviral effect in vitro.

Since Isatis root polysaccharide component G2 has no inhibitory effect on other respiratory viruses (non-influenza viruses), it shows specificity for inhibiting influenza viruses.

1. A process in preventing and/or treating diseases caused by influenza viruses and complications thereof, using Isatis root polysaccharide, wherein the molecular weight of the Isatis root polysaccharide is 3000-7000 Dalton.

2. The process according to claim 1, wherein the Isatis root polysaccharide is extracted from Radix Isatidis raw materials according to a method comprising:
A. decocting the Radix Isatidis raw materials by adding suitable times of distilled water to produce a decocted fluid, concentrating the decocted fluid to produce a concentrated solution so that the baume degree of which is 18-20° Bé at 50° C.;
B. cooling the concentrated solution obtained in Step A to below 45° C., adding alcohol to produce an alcohol solution so that alcohol content is up to 60% or above 60% in the alcohol solution, allowing the alcohol solution to stand for more than 12 hours to precipitate;
C. passing the alcohol solution through a macroporous resin column, eluting with pure water, and collecting an eluent;
D. deproteinizing the eluent, and placing the deproteinized solution in a dialysis bag with a proper molecular weight to dialyse; and
E. evaporating the solution inside and/or outside the dialysis bag with a rotary evaporator to concentrated solution, and then freeze-drying the resulted components with a vacuum freeze drier to obtain components of the Isatis root polysaccharide in powder form.

3. The process according to claim 1, wherein the influenza viruses are at least one member selected from the group consisting human influenza A, human influenza B, human influenza virus subtypes and H3N2, and avian influenza virus subtypes H6N2, H7N3, and H9N2.

4. A process in the manufacture of medicaments for preventing and/or treating diseases caused by influenza viruses and complications thereof, using Isatis root polysaccharide, wherein the molecular weight of the Isatis root polysaccharide is 3000-7000 Dalton, and the influenza viruses are at least one member selected from the group consisting human influenza A, human influenza B, human influenza virus subtypes H1N1 and H3N2, and avian influenza virus subtypes H6N2, H7N3, and H9N2.

5. The process according to claim 4, wherein dosage form of the medicament is a member selected from the group consisting of aerosols, capsules, ear drops, eye drops, eye ointments, gels, granules, injections, liniments, lotions, nasal drops, ointments, oral preparations, patches, pellicles, powders, solutions, suppositories, syrups, tablets, and tinctures.

6. A process in the manufacture of health foods or nutrition preparations for preventing diseases caused by influenza viruses and complications thereof, using Isatis root polysaccharide, wherein the molecular weight of the Isatis root polysaccharide is 3000-7000 Dalton; and the influenza viruses are at least one member selected from the group consisting human influenza A, human influenza B, human influenza virus subtypes H1N1 and H3N2, and avian influenza virus subtypes H6N2, H7N3, and H9N2.

7. A pharmaceutical composition for preventing and/or treating diseases caused by influenza viruses and complications thereof, comprising Isatis root polysaccharide with the molecular weight of 3000-7000 Dalton; and a dosage form, wherein the influenza viruses are at least one member selected from the group consisting human influenza A, human influenza B, human influenza virus subtypes H1N1 and H3N2, and avian influenza virus subtypes H6N2, H7N3, and H9N2.

8. The pharmaceutical composition according to claim 7, further comprising one or more pharmaceutically acceptable carriers or excipients; wherein the dosage form is a member selected from the group consisting of aerosols, capsules, ear drops, eye drops, eye ointments, gels, granules, injections, liniments, lotions, nasal drops, ointments, oral preparations, patches, pellicles, powders, solutions, suppositories, syrups, tablets, and tinctures, and the carriers or excipients in the pharmaceutical composition are selected from at least one member from the group consisting of diluents, adhesives, disintegrants, lubricants, substrates, aromatics, sweeteners, colorants, preservatives, antioxidants, coating agents, film-forming materials, solvents, solubilizers, wetting agents, adsorbents, filter aids, emulsifiers, surfactants, suspending agents, thickeners, plasticizer, chelating agents, transdermal enhancers, aerosol propellants, foaming agents, acidifying or alkalizing agents, and buffers.

9. A method for preparing the Isatis root polysaccharide, comprising:
A. decocting the Radix Isatidis raw materials by adding suitable times of distilled water to produce a decocted fluid, concentrating the decocted fluid to produce a concentrated solution so that the baume degree of which is 18-20° Bé at 50° C.;
B. cooling the concentrated solution obtained in Step A to below 45° C., adding alcohol to produce an alcohol solution so that alcohol content is up to 60% or above 60% in the alcohol solution, allowing the alcohol solution to stand for more than 12 hours to precipitate;
C. passing the alcohol solution through a macroporous resin column, eluting with pure water, and collecting an eluent;
D. deproteinizing the eluent, and placing the deproteinized solution in a dialysis bag with a proper molecular weight to dialyse; and
E. evaporating the solution inside and/or outside the dialysis bag with a rotary evaporator to concentrated solution, and then freeze-drying the resulted components with a vacuum freeze drier to obtain components of the Isatis root polysaccharide with proper molecular weight.

10. The method according to claim 9, further comprising:
A. decocting the Radix Isatidis raw materials by adding 8 times amount of distilled water for 2 hours at the first time to obtain a decocted fluid A and draff of Radix Isatidis; filtering the decocted fluid A and pouring into another container, adding 8 times amount of water into the draff and continue to decoct for 4 hours to obtain a decocted fluid B of Radix Isatidis;
B. filtering the decocted fluid B to produce a filtrate, and combining the filtrate with the filtrate of the decocted fluid A, concentrating to concentrated solution of Radix Isatidis, determining the baume degree of the concentrated solution with a baume hydrometer at 50° C., which is 18-20° Bé, and then cooling;
C. cooling the concentrated solution of Radix Isatidis to a temperature below 45° C., adding alcohol to produce an alcohol solution so that alcohol content is up to 60% or above 60% in the alcohol solution, allowing the alcohol solution to stand for more than 12 hours to precipitate;
D. passing the alcohol solution through a macroporous resin column, eluting with pure water, and collecting an eluent;
E. deproteinizing the Isatis root crude polysaccharide solution in pure water using sevage method: based on the property that proteins will denature in the organic solvent such as chloroform, shaking the mixture of sevag reagent and Isatis root crude polysaccharide solution in pure water vigorously with the ratio of sevag reagent (chloroform: n-butyl alcohol=5:1): Isatis root crude polysaccharide being 5:1 for 20 to 30 min to allow the proteins and chloroform-n-butyl alcohol generating gel so as to isolate the proteins from the Isatis root crude polysaccharide solution in pure water, and then centrifuging (4000 rpm/min×10 min) to remove the denatured proteins at the interface of aqueous layer and solvent layer, repeating for 5 times until there is no obvious denatured proteins precipitating out;
F. placing the aqueous layer in a dialysis bag with different molecular weight and then placing the dialysis bag holding the Isatis root crude polysaccharide solution in flask, adding tri-distilled water, and stirring for 24 hours with a magnetic stirrer; and
G. evaporating the solution inside and/or outside the dialysis bag with a rotary evaporator to concentrated solution, and then freeze-drying the resulted components with a vacuum freeze drier to powder to obtain the powder components of the Isatis root crude polysaccharide with proper molecular weight.

11. A method for preparing the pharmaceutical composition according to claim 7, comprising: using the Isatis root polysaccharide with a molecular weight of 3000-7000 Dalton as an active ingredient; adding auxiliary materials according to pharmaceutical formulations; and preparing preparations using the conventional technique in pharmaceutics.

12. A method for treating diseases caused by influenza viruses and complications thereof, comprising: administering therapeutically effective amount of pharmaceutical composition in claim 7; or administering therapeutically effective amount of Isatis root polysaccharide to the subject; wherein the molecular weight of the Isatis root polysaccharide is 3000-7000 Dalton.
